(19) United States
(12) Patent Application Publication
(10) Pub. No.: US 2006/0165804 A1
(43) Pub. Date: Jul. 27, 2006

(54) Polymer compositions for administration to animals

(76) Inventors: Andrew Lennard Lewis, Surrey (GB); Richard Redman, Surrey (GB); Pedro Garcia, Surrey (GB)

Correspondence Address: SUGHRUE MION, PLLC, 2100 PENNSYLVANIA AVENUE, N.W., SUITE 800, WASHINGTON, DC, 20037, US

(21) Appl. No.: 10/528,829

(22) PCT Filed: Sep. 23, 2003
(86) PCT No.: PCT/GB03/04075

§ 371 Date: Mar. 23, 2005

(30) Foreign Application Priority Data
Sep. 25, 2002 (EP) 02256647.5

(51) Int. Cl. A61K 31/77; A61K 9/14

(52) U.S. Cl. 424/489; 424/78.3

(57) ABSTRACT

Particulate embolotherapeutic compositions are described in which the particles comprise a matrix polymer expressing at surfaces available for interaction with biological molecules or, usually, blood cells, express zwitterionic groups, usually phosphoryl choline groups. The particles have suitable size and compressibility, for instance controlled by controlling the water content, for use in embolotherapy, by administration using catheters or hypodermic needles. The matrix polymer may be crosslinked. Preferably the particles are spherical. Examples are crosslinked polyvinylalcohol matrices coated with a crosslinkable copolymer formed of 2-methacryloyloxyethyl-2′-trimethylammoniumethyl phosphate inner salt with nonionic and crosslinkable comonomers. The coating is stabilised by crosslinking after coating from an organic solvent solution.

The present invention relates to polymer compositions for administration to animals, usually humans. The polymer is in particulate water-imbibed form during administration, and is preferably used to embolise body passageways. The method is preferably for therapy but may be for diagnosis.

Embolotherapy is a growing area of interventional medicine that normally relies upon the transarterial approach of a catheter to a desired location whereupon an agent is released in order to occlude a particular vessel. This treatment has been used in order to block the blood supply to certain hypervascularised tumours such as hepatocellular carcinoma, and more recently is becoming a popular choice of treatment for uterine fibroids. It is also used to treat abnormal shunts between arteries and veins known as arteriovenous malformations (AVMs), for venous malformations, to prevent post-operative bleeding etc.

There are a number of approved embolization devices including pushable coils, detachable coils and electolytically detachable coils. Also there is a line of detachable balloon products. There is also a range of embolic materials in use, that require transcatheter delivery to the site of embolization, whereupon they are released into the blood stream to block it. This is achieved either by a physical blocking of the vessel using small particles or spheres (usually aided by formation of thrombus), or in the case of liquid embolic agents, require some sort of phase change or reaction to set the flowable material and form a cast of the vessel.

The most popular particulate-based embolic agent is polyvinyl alcohol (PVA) foam particles, which has been used since the early 1970's. This material is irregular shaped and it can aggregate and form a blockage in the microcatheters commonly used for embolisation. Furthermore, in some instances the PVA particles are also known to not embolise as distally as expected based on its size. Although it has been approved for intravascular use for many years, PVA is not an adequately biocompatible material. It is reactive towards platelets, and some have worked on modifying the PVA with heparin to reduce this effect (Sefton, M. V. et al., Biomaterials, 1992; 13(7): 421-4). Indeed, the formation of thrombus contributes towards the embolic potential of the PVA particles, a large proportion of an embolised vessel often being filled with thrombus. This has the disadvantage that a patient that may be coagulopathic may not thrombose easily when PVA is used. Additionally, autolysis of the thrombus over time may result in unwanted recanalisation of the target vessel. PVA also causes moderate inflammation that takes several months to resolve. This may be a potential cause of long-term pain and complication. In some instances in animal studies, embolic agents that cause inflammation have been seen to be “ejected” from the vessel in much the same way as a splinter is removed from the skin (T. W. Clark, oral presentation: “Embolotherapy, materials, techniques and clinical applications”, Jun. 21-22, 2002, Pasadena, Calif.). Again, undesirable recanalisation can result in time periods that vary from days to several months.

Phosphorylcholine(PC)-based polymers are well known in the art. The only description for the use of phosphorylcholine-based polymers in embolotherapy has been in the form of polyionic complex gels in WO-A-0029481 and WO-A-0028920.

Particulate forms of PC polymers have been described. Ishihara has described the use of aggregates of PC polymers as drug carriers through the blood stream (Proc. Int. Symp. Controlled. Rel. Bioactive Mater., 1997, 24^{th}, 465466 and JP-A-11-322948). These aggregates however, are nano-sized by design, have excellent blood compatibility and are not intended to form an embolus.

Particle-containing columns are used to determine the thrombogenicity of materials. PC polymers have been coated onto cross-linked poly (methyl methacrylate) beads by solvent evaporation techniques for this purpose in the past (J. Biomed. Mater. Res., 24, 1069-1077, 1990). Poly(methyl methacrylate) is not water-absorbent.

Moreover, Sugiyama has reported extensively on the preparation of microspheres containing phosphorylcholine, normally made by emulsifier-free emulsion polymerisation in which the MPC stabilises the growing particle, and becomes incorporated into the sphere, and expressed at the surface. Spherical particles of polymer have been described made from styrene, methyl methacrylate, n-butyl methacrylate, hexyl methacrylate and styrene for example, with MPC contents from 0.1-10 mol % (Polymer Journal (Tokyo, Japan), 1994, 26(5), 561-569 Polymer J. 1993, 25(5), 521-527, and J. Polymer Sci A: Polym. Chem. 1997, 35, 3349-3357.). These acrylic/styrenic materials are relatively hard. They are synthesised by an oil-in-water emulsion polymerisation process so the polymer is unlikely to be water-swellable to a significant degree. The particle size of the product was less than 500 nm.

Zimehl et al, in Colloid Polym. Sci. (1990) 268, 924-933 describe oil-in-water emulsion polymerisation of polystyrene using potassium peroxodisulphate in the presence of N-(3-sulphopropyl)-N-methacrylomidyl propyl (N,N-dimethylammonium betaine) (SPP) at SPP concentrations in the range 5 to 70% by weight based on total monomer. The particle size of the latex product was dependent upon the initiator and the level of betaine comonomer. Again, all of the monomers were dispersed into the aqueous continuous phase before polymerisation was initiated, The solids concentration of product latex was around 10% by weight.

In U.S. Pat. No. 3,497,482 Hwa et al describe a copolymerisation of N,N-dimethyl-N-(2-methacryloyloxyethyl)-N-(3-sulphopropyl ammonium inner salt) (SPE), with ethyl methacrylate and acrylamide, in an aqueous continuous phase. Hwa produces a metastable oil-in-water product. He does not describe the particle size of the latex.

In WO-A-93/01221 we describe a range of copolymers of zwitterionic monomer with comonomers such as hydrophobic comonomers, ionic comonomers or reactive comonomers. The copolymers are formed by co-dissolving all the monomers into a solvent in which monomers and polymer are soluble and recovered by precipitation techniques. It is suggested that the product might be a micro emulsion but no examples of emulsion polymerisation are given.

According to the present invention there is provided a new therapeutic or diagnostic composition comprising particles of a polymer matrix into which is absorbed aqueous liquid, the particles having diameters in the range 40 to 4000 μm, characterised in that surfaces of the particles express zwitterionic groups.

The composition may be used for embolotherapy. The embolotherapy may be uterine fibroid embolisation, embolisation of blood vessels surrounding tumours or tumour excision sites, embolisation of varicose veins or varicoceles, arteriovenous malformations or venous malformations, and haemostasis of gastrointestinal bleeds and for embolisation pre- and post-resection. Alternatively the therapy may be for is filling aneurysms, packing tissue, wound healing, closing fistulas, or for othher medical or cosmetic uses such as sphincter packing applications. Diagnostic uses for the compositions may include monitoring the circulation.

The polymer matrix may be biodegradable, for instance under the conditions following administration. Biodegradable compositions may be useful where embolisation is required to be temporary. Preferably, however, the polymer matrix is substantially non-biodegradable under the conditions following administration. Where the composition is substantially non-biodegradable, it may be used for long term embolus therapy or tissue bulking, for instance. Preferably the polymer should not degrade under in vivo conditions, or simulated conditions, for a period of at least two years. A suitable test method may be devised. Biodegradability is known to be effected by the nature of the polymer, and the extent of any crosslinking, or other derivatisation. These may be selected by a person skilled in the art based upon the description below.

Although the compositions of the invention may comprise aqueous liquid only as the imbibed component in the particles, preferably the compositions further comprise a continuous aqueous medium in an amount sufficient to suspend the particles. The aqueous liquid is preferably physiological saline, optionally buffered to a physiological pH. Preferably the composition is suitable for introduction into the body without further preparation, and is thus sterile. It may be provided in a vessel from which it may be administered directly into the body, for instance by injection through a catheter or hypodermic needle.

Preferably the zwitterionic group is ammonium, phosphonium, or sulphonium phosphate or phosphonate ester zwitterionic group, more preferably a group of the general formula II

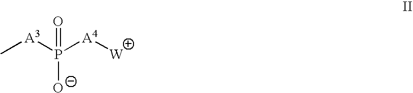

in which the moieties A³ and A⁴, which are the same or different, are —O—, —S—, —NH— or a valence bond, preferably —O—, and W⁺ is a group comprising an ammonium, phosphonium or sulphonium cationic group and a group linking the anionic and cationic moieties which is preferably a C₁₋₁₂-alkanediyl group,

preferably in which W⁺ is a group of formula

—W¹—N⁺R³₃, —W¹—P⁺R⁴₃, —W¹—S⁺R⁴₂ or —W¹-Het⁺ in which:

W¹ is alkanediyl of 1 or more, preferably 2-6 carbon atoms optionally containing one or more ethylenically unsaturated double or triple bonds, disubstituted-aryl (arylene), alkylene arylene, arylene alkylene, or alkylene aryl alkylene, cycloalkanediyl, alkylene cycloalkyl, cycloalkyl alkylene or alkylene cycloalkyl alkylene, which group W¹ optionally contains one or more fluorine substituents and/or one or more functional groups; and

either the groups R³ are the same or different and each is hydrogen or alkyl of 1 to 4 carbon atoms, preferably methyl, or aryl, such as phenyl, or two of the groups R³ together with the nitrogen atom to which they are attached form an aliphatic heterocyclic ring containing from 5 to 7 atoms, or the three groups R³ together with the nitrogen atom to which they are attached as heteroaromatic ring having 5 to 7 atoms, either of which rings may be fused with another saturated or unsaturated ring to form a fused ring structure containing from 5 to 7 atoms in each ring, and optionally one or more of the groups R³ is substituted by a hydrophilic functional group, and

the groups R⁴ are the same or different and each is R³ or a group OR³, where R³ is as defined above; or

Het is an aromatic nitrogen-, phosphorus- or sulphur-, preferably nitrogen-, containing ring, for example pyridine.

Generally a group of the formula II has the preferred general formula III 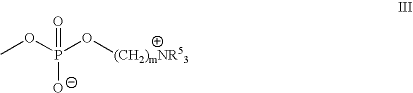
where the groups R⁵ are the same or different and each is hydrogen or C₁₋₄ alkyl, and m is from 1 to 4, in which preferably the groups R⁵ are the same preferably methyl.

In phosphobetaine based groups, X may have the general formula IV 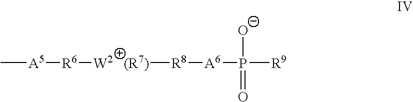
in which A⁵ is a valence bond, —O—, —S— or —NH—, preferably —O—;

R⁶ is a valence bond (together with A⁵) or alkanediyl, —C(O)alkylene- or —C(O)NH alkylene preferably alkanediyl, and preferably containing from 1 to 6 carbon atoms in the alkanediyl chain;

W² is S, PR⁷ or NR⁷;

the or each group R⁷ is hydrogen or alkyl of 1 to 4 carbon atoms or the two groups R⁷ together with the heteroatom to which they are attached form a heterocyclic ring of 5 to 7 atoms;

R⁸ is alkanediyl of 1 to 20, preferably 1 to 10, more preferably 1 to 6 carbon atoms;

A⁶ is a bond, NH, S or O, preferably O; and

R⁹ is a hydroxyl, C₁₋₁₂ alkyl, C₁₋₁₂ alkoxy, C₇₋₁₈ aralkyl, C₇₋₁₈-aralkoxy, C₆₋₁₈ aryl or C₆₋₁₈ aryloxy group.

In compounds comprising a group of the general formula IV, it is preferred that

A⁵ is a bond;

R⁶ is a C₂₋₆ alkanediyl;

W² is NR⁷:

each R⁷ is C₁₋₄ alkyl;

R⁸ is C₂₋₆ alkanediyl;

A⁶ is O; and

R⁹ is C₁₋₄alkoxy.

Alternatively X may be a zwitterion in which the anion comprises a sulphate, sulphonate or carboxylate group.

One example of such a group is a sulphobetaine group, of the general formula XI 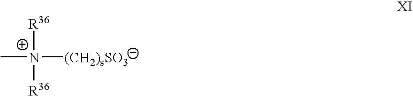
where the groups R³⁶ are the same or different and each is hydrogen or C₁₋₄, alkyl and s is from 2 to 4.

Preferably the groups R³⁶ are the same. It is also preferable that at least one of the groups R³⁶ is methyl, and more preferable that the groups are both methyl.

Preferably s is 2 or 3, more preferably 3.

Another example of a zwitterionic group having a carboxylate group is an amino acid moiety in which the alpha carbon atom (to which an amine group and the carboxylic acid group are attached) is joined through a linker group to the backbone of the biocompatible polymer. Such groups may be represented by the general formula XII

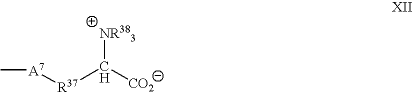

in which A⁷ is a valence bond, —O—, —S— or —NH—, preferably —O—,

R³⁷ is a valence bond (optionally together with A⁷) or alkanediyl, —C(O)alkylene- or —C(O)NHalkylene, preferably alkanediyl and preferably containing from 1 to 6 carbon atoms; and

the groups R³⁸ are the same or different and each is hydrogen or alkyl of 1 to 4 carbon atoms, preferably methyl, or two or three of the groups R³⁸, together with the nitrogen to which they are attached, form a heterocyclic ring of from 5 to 7 atoms, or the three group R³⁸ together with the nitrogen atom to which they are attached form a fused ring heterocyclic structure containing from 5 to 7 atoms in each ring.

Another example of a zwitterion having a carboxylate group is a carboxy betaine —N^{⊕}(R³⁹)₂(CH₂)ᵣCOO^{⊖} in which the R³⁹ groups are the same or different and each is hydrogen or R₁₋₄ alkyl and r is 2 to 6, preferably 2 or 3.

The zwitterionic groups may be components of a coating material, or may otherwise be attached to the surface of the polymer matrix, of which the polymer is formed of a non-zwitterion-containing polymer. For instance coatings may comprise polymers absorbed onto the surface of a matrix polymer. Such coatings should be stable under the conditions of use, for instance by being covalently bonded to the surface or, where the coatings comprise polymers, crosslinked at the surface. Alternatively, the polymer may be electrostatically bound to matrix polymer. The zwitterionic groups may be groups on the or one of the matrix polymers. For instance the matrix polymer may be a blend of a zwitterion group containing polymer and a second polymer which is different from the zwitterion group containing polymer.

Generally the zwitterionic groups are pendant groups on a polymer, either the matrix polymer or a coating polymer, formed from ethylenically unsaturated monomers including a monomer of the general formula I
YBX I
in which Y is an ethylenically unsaturated group selected from H₂C═CR—CO-A-, H₂C═CR—C₆H₄-A¹-, H₂C═CR—CH₂A², R²O—CO—CR═CR—CO—O, RCH═CH—CO—O—, RCH═C(COOR²)CH₂—CO—O,

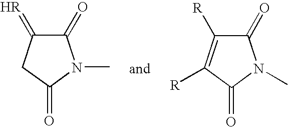

A is —O— or NR¹;

A¹ is selected from a bond, (CH₂)_{I}A² and (CH₂)_{I} SO₃— in which I is 1 to 12;

A² is selected from a bond, —O—, O—CO—, CO—O, CO—NR¹—, —NR¹—CO, O—CO—NR¹—, NR¹—CO—O—;

R is hydrogen or C₁₋₄ alkyl;

R¹ is hydrogen, C₁₋₄₋ alkyl or BX;

R² is hydrogen or C₁₋₄ alkyl;

B is a bond, or a straight branched alkanediyl, alkylene oxaalkylene, or alkylene (oligooxalkylene) group, optionally containing one or more fluorine substituents;

X is the zwitterionic group.

In the zwitterionic monomer of the general formula I it is preferred that the ethylenic unsaturated group Y is H₂C═CR—CO-A-. Such acrylic moieties are preferably methacrylic, that is in which R is methyl, or acrylic, in which R is hydrogen. Whilst the compounds may be (meth)acrylamido compounds (in which A is NR¹), in which case R¹ is preferably hydrogen, or less preferably, methyl, most preferably the compounds are esters, that is in which A is O.

In monomers of the general formula I, especially where Y is the preferred (alk)acrylic group, B is most preferably an alkanediyl group. Whilst some of the hydrogen atoms of such group may be substituted by fluorine atoms, preferably B is an unsubstituted alkanediyl group, most preferably a straight chain group having 2 to 6 carbon atoms.

Monomers in which X is of the general formula II in which W⁺ is W¹N^{⊕}R³₃ may be made as described in our earlier specification WO-A-9301221. Phosphonium and sulphonium analogues are described in WO-A-9520407 and WO-A-9416749.

Monomers comprising a group of the general formula IV may be made by methods as described in JP-B-03-031718, in which an amino substituted monmoer is reacted with a phospholane.

A particularly preferred zwitterionic monomer is 2-methacryloyloxyethyl-2′-trimethylammonium ethyl phosphate inner salt.

The ethylenically unsaturated monomers may further comprise comonomers, for instance compounds of the general formula V 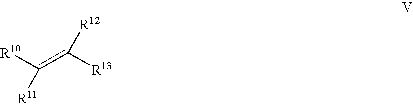
in which R¹⁰ is selected from hydrogen, halogen, C₁₋₄ alkyl and groups COOR¹⁴ in which R¹⁴ is selected from hydrogen and C₁₋₄ alkyl;

R¹¹ is selected from hydrogen, halogen and C₁₋₄ alkyl;

R¹² is selected from hydrogen, halogen, C₁₋₄ alkyl and groups COOR¹⁴ provided that R¹⁰ and R¹² are not both COOR¹⁴; and

R¹³ is a C₁₋₁₀ alkyl, a C₁₋₂₀ alkoxycarbonyl, a mono-or di-(C₁₋₂₀ alkyl) amino carbonyl, a C₆₋₂₀ aryl (including alkaryl) a C₇₋₂₀ aralkyl, a C₆₋₂₀ aryloxycarbonyl, a C₁₋₂₀-aralkyloxycarbonyl, a C₆₋₂₀ arylamino carbonyl, a C₇₋₂₀ aralkyl-amino, a hydroxyl or a C₂₋₁₀ acyloxy group, any of which may have one or more substituents selected from halogen atoms, alkoxy, oligo-alkoxy, aryloxy, acyloxy, acylamino, amine (including mono and di-alkyl amino and trialkylammonium in which the alkyl groups may be substituted), carboxyl, sulphonyl, phosphoryl, phosphino, (including mono- and di- alkyl phosphine and tri-alkylphosphonium), zwitterionic, hydroxyl groups, vinyloxycarbonyl and other vinylic or allylic substituents, and reactive silyl or silyloxy groups, such as trialkoxysilyl groups;

or R¹³ and R¹² or R¹³ and R¹¹ may together form —CONR¹⁵CO in which R¹⁵ is a C₁₋₂₀ alkyl group.

It is preferred for at least two of the groups R¹⁰R^{11,}R¹² and R¹³ to be halogen or, more preferably, hydrogen atoms. Preferably R¹⁰ and R¹¹ are both hydrogen atoms. It is particularly preferred that compound of general formula V be a styrene-based or acrylic based compound. In styrene based compounds R¹³ represents an aryl group, especially a substituted aryl group in which the substituent is an amino alkyl group, a carboxylate or a sulphonate group. Where the comonomer is an acrylic type compound, R¹³ is an alkoxycarbonyl, an alkyl amino carbonyl, or an aryloxy carbonyl group. Most preferably in such compounds R¹³ is a C₁₋₂₀-alkoxy carbonyl group, optionally having a hydroxy substituent. Acrylic compounds are generally methacrylic in which case R¹² is methyl.

Preferably the comonomer is a non-ionic comonomer, such as a C₁₋₂₄ alkyl(alk)-acrylate or -acrylamide, mono- or di-hydroxy-C₁₋₆-alkyl(alk)-acrylate, or -acrylamide, oligo(C₂₋₃ alkoxy) C₂₋₁₈-alkyl (alk)-acrylate, or -acrylamide, acrylamide styrene, vinylacetate or N-vinyllactam.

Where the zwitterionic groups are in the form of a coating on a matrix polymer, the polymerisation of the ethylenically unsaturated monomers may be carried out in the presence of matrix polymer, for instance as a graft polymerisation process as described in our earlier application number WO93/05081. Alternatively, the monomers including zwitterionic monomer may be preformed into a polymer and then combined with matrix polymer, generally by coating onto the surface of preformed matrix polymer. Stable binding polymers suitable for use as coating polymers, comprising zwitterionic groups, are described in our earlier publication WO93/01221.

Where the zwitterionic groups are on matrix polymer the matrix polymer may consist only of the zwitterionic group containing polymer, or may comprise a mixture of the zwitterion-groups containing polymer and a second, different polymer. The second polymer and zwitterionic group containing polymer may be mixed after polymerisation and may or may not be cross-linked to one another. One of the polymers may be formed by polymerisation in the presence of the other, preformed polymer. Examples of blends of zwitterion groups containing polymers and other polymers are given in WO-A-9414897 and blends which have appropriate water-absorbing, water-insoluble and compressible properties may be selected from the blends described in that document and formed into particles. Polyion complexes may be considered as blends.

The matrix polymer may be substantially non-crosslinked. For instance polymers having a proportion of hydrophobic moieties may be rendered water-insoluble by the presence of such moieties, but nevertheless be capable of absorbing aqueous liquids so as to be useful in the invention. Such matrix polymers may be formed of ethylenically unsaturated monomers including hydrophobic monomers such as alkyl(alk) acrylates or -acrylamides or, less preferably, styrene. Preferably, however, the matrix polymer is crosslinked. This allows the formation of particles which have adequate mechanical stability, water-swellability and cohesiveness to be useful in the invention. Crosslinking may be electrostatic. For instance a polymer having several charges of one polarity may be crosslinked using a counterionically charged di- or higher-valent crosslinker, generally a polymer having pendant charges of the opposite polarity. Blends of counterionically charged polymers may be termed polyion complexes.

One specific embodiment of a polymer matrix which is electrostatically crosslinked, is a polyion complex formed of a first polymer having pendant zwitterionic groups and pendant anionic or cationic groups, and a second polymer having pendant cationic or anionic groups, as the case may be, blended in amounts so as to give ratios of overall cationic:anionic charges in the range 2:1 to 1:2. Preferably the level of anionic and cationic charges is in the range 5 to 100% based on repeat units of the polymer. Such polyion complexes may be formed by combining the two polymers so as to form a blend, in the presence of a solvent, and then removing the solvent. Suitable processes, appropriate for forming water-swellable, water-insoluble blends are described in our earlier publication number WO00/29481.

Preferably the matrix polymer is covalently crosslinked, since such particles have a higher mechanical stability. Where the matrix polymer is formed from ethylenically unsaturated monomers, crosslinking may be by including in the ethylenically unsaturated monomers a di- or multi-ethylenically unsaturated monomer which forms crosslinks during the polymerisation process. A multi-valent crosslinker may be a macromer, that is a polymer having terminal or, preferably, pendant ethylenically unsaturated groups. Cross-linked polymers formed by polymerising zwitterionic monomers with cross-linking monomers and non-ionic comonomers which are suitable to form particles for use in the present invention are described in WO-A-92. Alternatively, the ethylenically unsaturated monomers may comprise one or more monomers having pendant functional groups, capable of forming intermolecular crosslinks, during or, more preferably, subsequent to the polymerisation step. Such functional monomers may be any of those defined as being capable of forming covalent bonds with underlying surfaces, or intermolecular crosslinks, in our earlier publication WO93/01221 or WO98/30615.

As mentioned above, a commercially available particulate embolotherapeutic composition comprises crosslinked particles of polyvinyl alcohol. One product, available under the trade name Drivalon, is said to comprise spheroidal particles which swell in the presence of aqueous liquid, the matrix polymer being polyvinyl alcohol crosslinked by an aldehyde. Another type of product comprises foamed polyvinyl alcohol which has an irregular external shape, the dimensions of which change little between its dry state and its water-imbibed state. Again the polyvinyl alcohol is believed to be crosslinked with an aldehyde, such as glutaraldehyde. Another polyvinyl alcohol-based particulate composition has been described in WO01/68720. The particles are formed by crosslinking prepolymerised polyvinyl alcohol which has been modified by binding of ethylenically unsaturated pendant groups. The reaction to form the pendant groups is described in earlier US publication number 5508317 and publications related thereto by common priority. The ethylenically unsaturated pendant groups are crosslinked by a radical initiated reaction whereby addition reactions take place between intermolecular ethylenically unsaturated groups, to render the polyvinyl alcohol water-insoluble but water-swellable. Particles may be formed either by comminuting the cross-linked product as a bulk solid, or by forming particle precursors by dispersing the derivatised polyvinyl alcohol into a non-solvent to form a dispersed phase in which crosslinking is initiated. The particles may be recovered from the non-solvent, for instance a non polar organic solvent. Aldehyde-based crosslinking of polyvinyl alcohol in a dispersed phase may also be used to form particulate materials by the method described by Thanoo et al, J. App. Biomat. (1991), 2, 67 to 72.

Covalently crosslinked polyvinyl alcohols may be provided with zwitterionic surfaces by coating preformed particles with zwitterion containing coating polymers so as to form a stable coating. Coating processes and suitable polymers are, for instance, described in our earlier publication WO93/01221 and WO98/30615. The coating may be carried out with the preformed crosslinked polyvinylalcohol particles in a water-imbibed or dry form. The coating may be carried out with the particles in a particulate form (i.e. without a continuous liquid medium in which the particles are suspended) or, preferably, may be carried out in a liquid suspending medium comprising the coating polymer. Suitable worked examples are described below.

Other cross-linked polymers which have been used to form particulate embolotherapeutic compositions are cross-linked hyaluronic acid and other polysaccharides. These polymers are also suitable as matrix polymers.

In the present invention, the zwitterionic groups should be expressed at the surface, by which we mean that surfaces which are exposed to biological liquids during use should carry zwitterionic groups. The effect of the zwitterionic groups is that the surfaces are rendered non-thrombogenic. It is thus generally the surfaces, that would be accessible to the components that lead to thrombogenicity, and particularly to blood cells themselves, which carry the Zwitterionic groups. Where the polymer matrix is substantially non-porous, that is does not have pores large enough to allow access by blood cells, or even by large biomolecules, the particles may have zwitterionic groups only on their peripheral surfaces. However where the matrix is porous, for instance a polyvinyl alcohol sponge-type material, it is additionally the surfaces of the pores which are provided with zwitterionic groups. A coating process for a porous material must thus allow the coating material comprising zwitterionic groups, to contact the surfaces of the pores so that a coating is deposited thereon. A suitable process is exemplified below.

It is surprising in the invention that particles provided with zwitterionic groups so as to reduce thrombogenicity of the surfaces as compared to surfaces which are similar apart from being free of zwitterionic groups, produces compositions which are capable of forming an embolus. As mentioned above, the prior art has suggested that particulate embolotherapeutic compositions are effective partly by virtue of causing a thrombus where the particles lodge in the circulation. If and when the thrombus is broken down, in the normal course of events, recanalization may result. The present invention allows a longer lasting embolus to be generated which is not susceptible to early recanalization upon thrombolysis (autolysis).

A further advantage of the present compositions is that they are expected to minimise any inflammatory reaction at the site of the embolus. This should avoid the problems experienced with prior art compositions which are ejected from the site of the embolus by the inflammatory reaction of the body to their presence. This is believed to reduce the rate of recanalization which results from such a mechanism.

Although the particles may have an irregular shape, for instance formed by comminuting a bulk solid or gel of matrix polymer material, or by agglomerating or granulating small particles of matrix polymer, preferably the particles are substantially spherical in shape. It is believed that spherical particles will have a lower inflammatory or other adverse reaction by tissue at the site of implantation.

The size of the particles in the composition is selected upon the intended use and the delivery means. For instance, where the compositions are to be used in embolotherapy, the particle size is selected according to the diameter of the vessel to be embolised. As is known for particulate embolotherapy compositions currently available, it may be convenient for a range of compositions to be provided for use by the practitioner, each of which comprises particles within a different size range. Whilst the sizes in different members of a product range may overlap, it is generally convenient for the particles within the products to have been produced in a single process and to have been separated after production on the basis of size, whereby there is little overlap between the particle sizes.

The particle diameters may be measured by such techniques including the use of a Malvern Multisizer, digital image analysis, optical microscopy, and by use of calibrated graticules or by sieving. The particles of the invention have sizes above 40 μm for instance when the have absorbed physiological saline to equilibrium. Preferably the particles all when fully imbibed with water have diameters more than 100 μm, for instance more than 150 μm. Although the maximum size of the particles may be up to 4000 μm, preferably substantially all the particles have sizes less than 3000 μm, for instance up to 2000 μm. Where a product range comprises populations having different particle sizes, suitable size ranges are 40-100 μm, 100-300 μm, 300-500 μm, 500-700 μm, 700-900 μm, 900-1200 μm, 1200-1500 μm, 1500-2000 μm and 2000-2800 μm.

The present invention provides additionally the use of particles as defined above in relation to the first aspect of the invention, in the manufacture of a composition for administration to an animal for therapy or diagnosis.

In this aspect of the invention, the composition may be administered for forming an embolus, for instance in a method for uterine fibroid embolisation, embolisation of vessels around tumours or tumour-excision sites, embolisation of varicose veins or varicoceles, or of arteriovenous malformations or venous malformations, hemostasis of gastrointestinal bleeds, embolisation of fistulas, and embolisation of fallopian tubes and seminiferous tubes for sterilisation.

The composition which is administered to an animal, which is preferably a human, may be in the form of swollen gel particles without a continuous liquid phase. However preferably the composition comprises a continuous aqueous liquid which suspends the particles. The particles may be stabilised against sedimentation, for instance by the use of a continuous aqueous phase of the same density as the swollen particles (isobuoyant), by viscosifying the continuous phase, or by the use of stabilisers, for instance which coat the particles. The continuous aqueous liquid may be added to particles supplied in dry or partially imbibed form, immediately before administration to the animal. Preferably, however, the composition is provided ready for use, and is thus generally sterile, and may be provided in a vessel from which it may be directly administered, such as via a catheter, or a hypodermic needle.

Some of the polymer compositions themselves may be novel. They may have uses other than for direct introduction, or for making up into compositions suitable for administration, to an animal. According to a further aspect of the invention, there are provided novel microspheres comprising a core which is a matrix of a water-insoluble water-absorbing polymer, which when imbibed with physiological saline at equilibrium at room temperature have diameters in the range 40 to 4000 μm, characterised by expressing zwitterions over their external surfaces.

Processes for producing some embodiments of the novel compositions are also part of the invention and are claimed in claims 30-33.

In this aspect of the invention, the size distribution of the microspheres is such that the difference between the diameter of the smallest and the largest particles is between 50 μm and 1000 μm, preferably 100-500, more preferably 150-300 μm. A series of populations having different size ranges may be provided in a kit. The size ranges of the populations in such a kit may or may not overlap.

The microspheres are preferably for use in a method of treatment in an animal by therapy or diagnosis.

The compositions which are administered to an animal in any of the aspects of the present invention may comprise additional components, selected according to the application. The compositions may comprise imaging agents, for instance radiopaque agents or magnetic resonance contrast agents, allowing the location of the compositions to be monitored following administration. The imaging agents may be bound in the particles, or may be suspended in a continuous aqueous suspending liquid. For instance, known radiopaque contrast agents such as Visipaque, Omnipaque, Lipiodol or other commercially available materials may be admixed with the composition of the inventions before administration to the animal subject. Preferably the contrast agent is a non-ionic material. Alternatively, the contrast agent may be covalently bonded to the matrix polymer material or to a coating polymer, for instance by copolymerising an iodinated monomer such as 2-(2′-iodobenzoyl)ethyl methacrylate or 2-(2′,3′,5′-triiodobenzoyl)ethyl methacrylate with other ethylenically unsaturated monomers as described by Benzina, A. et al., Biomat. (1994)15(14) 1122-1128 and J. Biomed. Mater. Res. (1996) 32(3), 459-466. Alternatively the contrast agent may merely be dispersed through the polymer matrix, though not covalently bonded thereto, for instance by being dispersed with the components from which the particles are formed during production.

Additionally or alternatively, the compositions may have drugs present therein. The drug may be suspended in the aqueous continuous phase surrounding the particles and/or may be within the particles, either in solution in the aqueous liquid imbibed therein, by being covalently bonded to the matrix polymer, by being covalently or electrostatically bonded to the external surface or partitioned into hydrophobic domains. Where the drug is within the partible, it may be released over an extended period of time after administration to the animal subject. Drug delivery systems comprising particulate embolotherapeutic materials are known. Based on the common general knowledge concerning methods of incorporating drugs into particulate drug delivery systems of this type, and for controlling the release therefrom, the skilled person may put this embodiment of the invention into effect. Suitable drugs may include, for instance, antibiotics, thrombotic agents, where thrombosis is desired, and/or cytotoxic or cytostatic drugs, for chemoembolisation of tumours, for instance. The drug may be an anti-angiogenic drug. Microsphere delivery systems for delivery of anti-angiogenic drugs are described in WO95/03036. Any of the drugs described in that specification may be used in the present invention, especially for use in treating tumours or tumour excision sites.

The compositions are administered using the devices currently in use for delivery of particulate embolotherapy. The device may comprise a cathether or a hypodermic needle attached to a syringe. The device is selected according to the particle size of the composition being delivered and the particles' elastomeric properties. Thus the conduits through which the composition flows during delivery must be sized so as to allow passage of the particles without blockage.

The particles in their water-imbibed form, that is when being administered, have a degree of elasticity. During delivery when they are subjected to compressive forces they may therefore be deformed within the conduits of the device. Where the particles have a regular shape, especially where they are substantially spherical, the particles should recover after any such deformation. The elastic properties may be investigated by methods in which particles in their swollen form are subjected to predetermined pressure, at speeds intended to simulate the environment during delivery through the device, whilst measuring the extent of compression. The test may be conducted to the elastic limit, and/or may be reversed by removing pressure and measuring recovery. We have found that particles, which, when fully imbibed with water, i.e. left to equilibrate with physiological saline, have a water content of at least 30% by weight preferably at least 50% by weight, have good elastic properties. The invention is further illustrated in the accompanying examples.

### REFERENCE EXAMPLE 1

### Reduced Inflammation Using PC Materials

An in vitro method looking at the adhesion of mononuclear cells obtained from human blood, has been used to assess the potential immune response to a PC-based material:

24 mL of human venous blood, obtained from a 36 year old healthy male volunteer (one donor to eliminate problems associated with donor variation) was collected into a 30 mL universal tube (Bibby Sterilin, Staffordshire, UK) containing 12 μg of heparin (Sigma Aldrich, Poole, Dorset, UK) dissolved in 500 μL of sterile deionised water. The separation of blood was then carried out following the manufacturers methodology using Histopaque 1077 (Sigma, Poole, Dorset, UK): the heparinsed blood was divided into two and carefully layered onto 12.5 mL of Histopaque 1077 (Sigma Aldrich, Poole, Dorset, UK) in two clean universal centrifuge tubes. These tubes were then centrifuged (Denley, BS400) at 700 g for 30 minutes at room temperature (18-26° C.). Following centrifugation the distinct opaque/grey layer in each tube containing mononuclear cells were collected and transferred to a clean centrifuge tube. The cells were then washed by addition of 10 mL of sterile PBS followed by centrifugation for 10 minutes at 200 g at room temperature to wash the cells. Following centrifugation the supernatant was removed and the pelleted cells were resuspended in PBS, the washing procedure was then repeated twice. After washing, the cells were finally re-suspended in 10 mL of RPMI 1640 medium (Invitrogen, Paisley, UK) supplemented with 10% human serum, 1% penicillin and streptomycin (Sigma Aldrich, Poole, Dorset, UK), and 1% L-glutamine (Sigma Aldrich, Poole, Dorset, UK). The cells were then counted using a haemacytometer.

Poly(ethylene terephthalate) film was coated with a 5 g/l solution in ethanol of a quater polymer of 2-methacryloyloxy ethyl-2′-trimethylammonium ethyl phosphate inner salt (MPC):n-dodecyl methacrylate:hydroxypropylmethacrylate trimethoxysilylpropyl methacrylate 23:47:25:5 by mole synthesised as described in WO-A-9830615. The coating was dried then heated to cross-link the polymer at a temperature of 70° C. overnight.

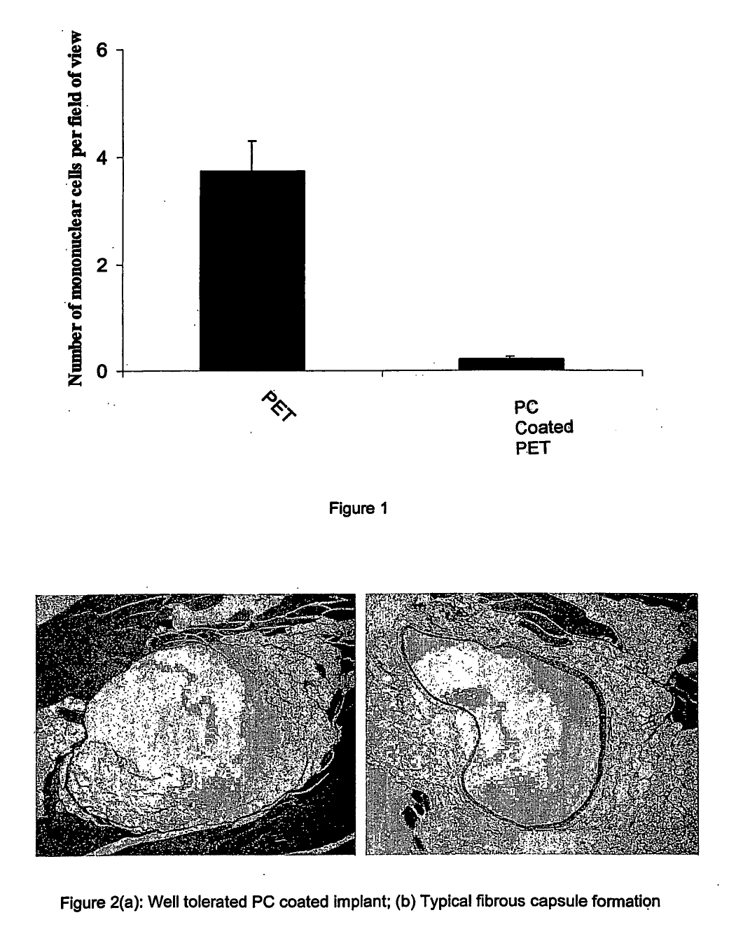

Cells were then seeded directly onto the test materials (poly(ethyleneterephthalate) (PET) and PC polymer coated PET cut into samples of 1 cm²) at 2.5×10⁵ cells per sample in a total volume of 400 μL of supplemented media. The concentration was chosen to be low enough to allow good visualisation of cell adhesion, but dilute enough to minimise the interaction between cells and prevent formation of cell aggregates. Plates were then incubated in a humidity incubator (Sanyo, Jencons PLS, UK) overnight at 37° C. and 5% CO₂. After incubation samples were washed three times with 0.5 mL aliquots of sterile PBS. Adhered cells were then fixed by the addition of 0.5 mL of 4% (w/v) paraformaldehyde (Sigma Aldrich, Poole, Dorset, UK) prepared in deionised water for twenty minutes at room temperature. The samples were then washed again three times in 0.5 mL aliquots of sterile PBS before incubation with 0.5 mL of 4% (w/v) BSA (Sigma Aldrich, Poole, Dorset, UK) prepared in sterile PBS, for 30 minutes at room temperature. The samples were then washed three times with 0.5 mL of sterile PBS. The cells were then permeabilised by the addition of 0.5 ml of 0.2% (v/v) Tritron X-100 (Sigma Aldrich, Poole, Dorset, UK) prepared in PBS and incubated for 8 minutes at room temperature. The samples were then washed in sterile PBS as before. 0.5 mL of 9% (v/v) hydrogen peroxide (Sigma Aldrich, Poole, Dorset, UK) prepared in deionised water, was added to each sample and incubated overnight at 4° C. Following incubation with hydrogen peroxide the samples were washed in sterile PBS. Samples were then placed on top of a piece of parafilm (BDH Laboratory Supplies, Poole, Dorset, UK) in humidity chambers (petri dish (Invitrogen, Paisley, UK) lined with filter paper (BDH Laboratory Supplies, Poole, Dorset, UK) soaked in sterile PBS). 50 μL of the CD68 (a macrophage specific cell surface receptor) primary antibody (DAKO, Cambridgeshire, UK) diluted 1:20 in PBS with 1% FCS (Invitrogen, Paisley, UK) was added to each sample and incubated for 1 hour at room temperature to prevent non-specific binding. After incubation the samples were dipped 10 times in 40 mL of PBS, this dipping process was repeated a twice in fresh PBS. A rapid stain kit (Sigma Aldrich, Poole, Dorset, UK) was then applied to the samples following the instruction supplied with the kit; one drop (˜50 μL) of biotinylated secondary antibody (Goat anti-mouse immunoglobulins in buffered saline and 0.1% sodium azide) was added to each sample and incubated at room temperature for 5 minutes. Samples were then washed as before in PBS. One drop (˜50 mL) of peroxidase reagent (conjugated in buffered saline) was then added to each sample and incubated for five minutes at room temperature. Samples were washed in PBS as before and 50 μL of substrate reagent (4 mL deionised water, 2 drops (˜100 μL) acetate buffer (2.5 M pH 5.0), 2 drops (˜100 μL) AEC chromogen (3-amino-9-ethylcarbazole in N,N-dimethylformamide) and 1 drop (˜50 μL) of 3% hydrogen peroxide (in deionised water)) was added to each sample. Samples were then washed again in PBS and finally in deionised water before mounting on microscope slides using about 20 μL Vectasheild mountant (Vectorlabs, Peterborough, UK). The number of adherent macrophage and monocyte cells were counted per field of view under ×400 magnification using a light microscope (Laborlux 12, Leitz, Germany). The average number of cells per field of view was calculated from thirty random fields of view per sample replicate. The results in FIG. 1 shows mean number of cells per field of view (obtained form 30 fields of view) and standard error of the mean obtained from 18 replicate samples as observed under ×400 magnification.

A significantly greater number of mononuclear cells were found adherent to PET compared to PC 1036 (Student's t test, p<0.05, n=18).

### REFERENCE EXAMPLE 2

### Reduced Fibrous Capsule Using PC Materials

Stainless steel rods coated with a cross-linkable PC polymer as used in Reference Example 1 above were implanted intramuscularly into rabbits and fibrous capsule formation compared to that obtained from PE negative control implants. Statistical analysis of the fibrous capsule formation around the implant at 13 weeks revealed less capsule for the PC coated implants, the sample often being surrounded by a mature adipose tissue, indicative that the implant was well-tolerated and accepted by the animal (FIGS. 2a & 2b):

Using the conventional scoring methods to define level of capsule formation, the implants were compared at 13 weeks (n=20 for each) and produced scores of 0.25 and 0.75 for the PC coated and control samples respectively (p=0.011). There was significantly less capsule present at 13 weeks for the PC coated sample.

The observations made in reference examples 1 and 2 support the claims of the invention that modification of the embolization agent with PC should help not only in the prevention of thrombus formation (which is well known and widely reported for PC materials), but also that the longer-term inflammatory response to the agent will be reduced.

### EXAMPLE 1

### Preparation of Cross-linked PC Polymer Hydrogel Embolisation Particles

A crosslinkable PC polymer having the components as the polymer used in Reference Example 1 was prepared and isolated as outlined in WO-A-9830615 and recovered by precipitation into acetone. The recovered material was dried and cross-linked in an oven at 70° C. for 4 hours. The particulate material was sorted into size ranges using a series of sieves (Christison, Gateshead UK, 63-830 μm, stainless steel, 200 mm diameter). When particles were hydrated in water be at room temperature to equilibrium they reached a water content in the range 50-55%.

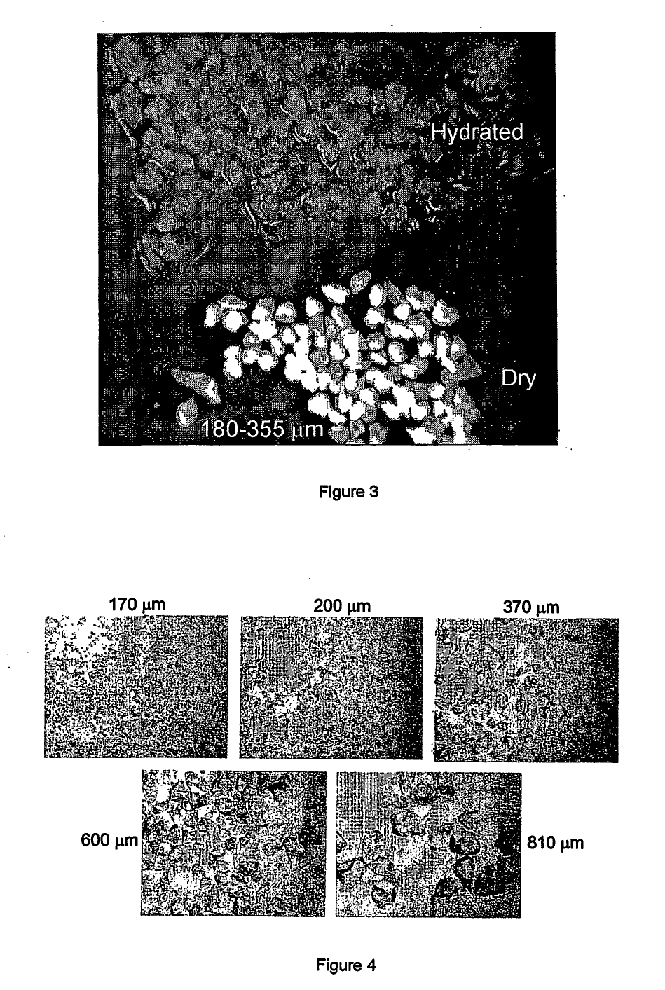

The micrographs in FIG. 3 show a sample of dry and a sample hydrated particles which had sieved sizes (dry sieved) in the range 180-355 μm. The water content of the hydrated particles was determined by hydrating them in physiological saline at room temperature for 24 h. A sample of the hydrated particles were blotted dry then weighed. The particles were then dried in an oven at 100° C. for 1 hr and reweighed. The difference in weight is the reported equilibrium water content. The particles of this example had equilibrium water contents of 50 to 55%.

### EXAMPLE 2

### Preparation of Cross-linked PC Polymer Hydrogel Embolisation Particles

An alternative crosslinked PC polymer was prepared by bulk copolymerisation of a solution of MPC in 2-hydroxy-ethylmethacrylate cross-linked with ethylene glycol dimethacrylate, in which the MPC is present in about 7 mole % and the cross-linker is present in an amount of about 0.5 mole %, generally as described in WO92/07885. The bulk xerogel material was milled into particulates and size-sorted using the sieving technique described in Example 1. FIG. 4 shows micrographs of xerogel (i.e. before equilibration in water) particles which are retained on sieves of the specified aperture. Upon hydration, the particles swelled rapidly to attain a water content of around 60%.

### EXAMPLE 3

### Preparation of PC-based Microspheres for Embolotherapy

0.25 g of ethyl cellulose (ethoxy content 48%, Aldrich) was added to a solution of 1009 of dichloromethane and 50 g of hexane in a stirred 500 ml reaction vessel. The solution was deoxygenated with nitrogen and the stirrer speed adjusted to 250 rpm.

In a separate vessel a solution was made up with 17.5 g dimethylacrylamide (Aldrich), 0.125 g of methylene bis acrylamide (Aldrich) and 45.0 g of distilled water. To this solution was added 7.5 g of MPC and 0.125 g of ammonium persulfate (Aldrich). This clear aqueous solution was then added to the stirred reaction vessel.

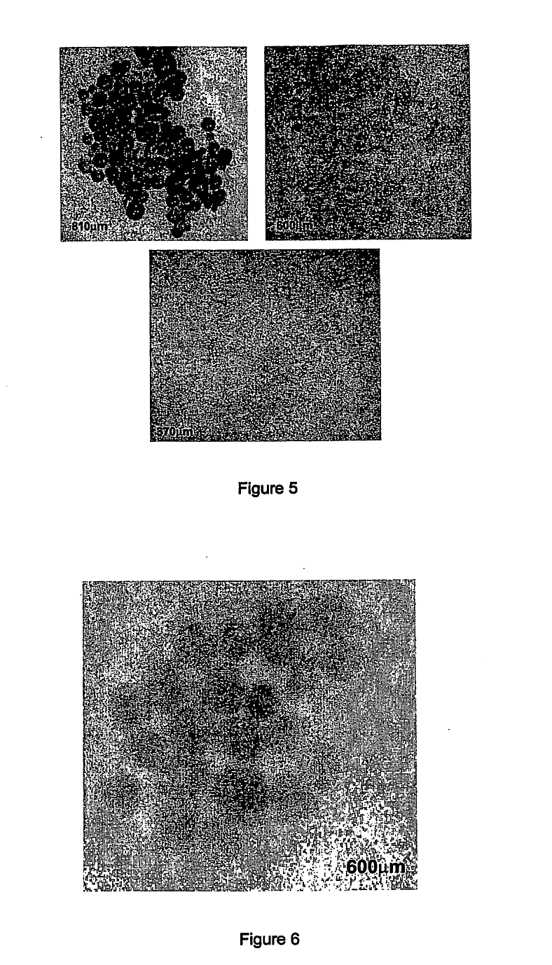

The total contents were deoxygenated for a further 10 minutes before adding 0.25 g of tetramethyl ethylene diamine (Aldrich). After the polymerisation was complete (ca. 10° C. exotherm) the reaction vessel is contents were poured through a ca. 2 mm sieve and the spherical particles removed from the solvents by filtration. The particles were further washed several times with acetone and distilled water, and stored in distilled water. The particles were separated into size ranges using a wet sieving method using a series of sieves having progressively decreasing apertures in the range 370 μm to 810 μm. FIGS. 5a-c show some representative spheres produced by this method the size in micrometers shown on the figure being the aperture of the sieve in which the particles were retained. The spheres are stained with various dyes, such as Eosin Y or Reactive Blue in order to visualise them more effectively in these micrographs. The dyes, which are water-soluble, in aqueous solution, are contacted with polymer for a suitable period and if necessary are then fixed by being subjected to conditions adapted to the reactive group on the dye, and the polymer.

### EXAMPLE 4

### Preparation of PC-based Microspheres by a UV Cross-Linking Method

A polymer was synthesised by a monomer-starved free radical polymerisation technique, containing 30 wt % MPC, 50 wt % lauryl methacrylate, 10 wt % hydroxpropyl methacrylate and 10 wt % allyl methacrylate in ethanol, with 1 wt % azobisisobutyronitrile (AIBN) as initiator. The solution was precipitated into acetone to yield 85% of a white polymer which was dried overnight at room temperature in vacuo.

7.5 g of the polymer and 0.1 g of Darocure UV initiator were dissolved in 100 g of chloroform with vigorous stirring. A 250 ml round bottomed vessel was fitted with an overhead stirrer and filled with 230 ml of deionised water to avoid excessive vortex formation. To the stirred water (150 rpm) was added 5 g of the polymer solution, which immediately emulsified to form droplets. The viscosity of the polymer solution was critical in determining the size and quality of the spherical droplets formed. Upon complete addition of all of the polymer solution, a UV lamp was switched on and the vessel irradiated for 10 minutes. The resulting droplets polymerised via radical polymerisation via radical polymerisation of the allyl pendant groups into a cross-linked PC polymer spheroids which were wet sieved to recover sample sizes in the range 300-900 μm. The spheres collected on a 600 μm aperture sieve are shown in Figure

### EXAMPLE 5

### Preparation of PC-based Polyionic Complex Embolisation Particles

Polymers were prepared according to the methods outlined in WO-A-0029481. Anionic and cationic polymers of the following formulae were prepared, dissolved in pure water to produce 17.5 wt % solutions of each, which were subsequently mixed thoroughly 1:1 by volume to obtain a PC polyion complex (PIC) gel of the same wt % composition: LMA is laurylmethacrylate (n-dodecyl) methacrylate and DMA is N,N-dimethylacrylamide.

Cationomer: MPC₂₅LMA₁₅TEM₆₀ (TEM=choline methacrylate chloride, Mw 207)

Anionomer: MPC₃₀DMA₁₀SSS₆₀ (SSS=Sodium styrene sulphonic acid, Mw 207)

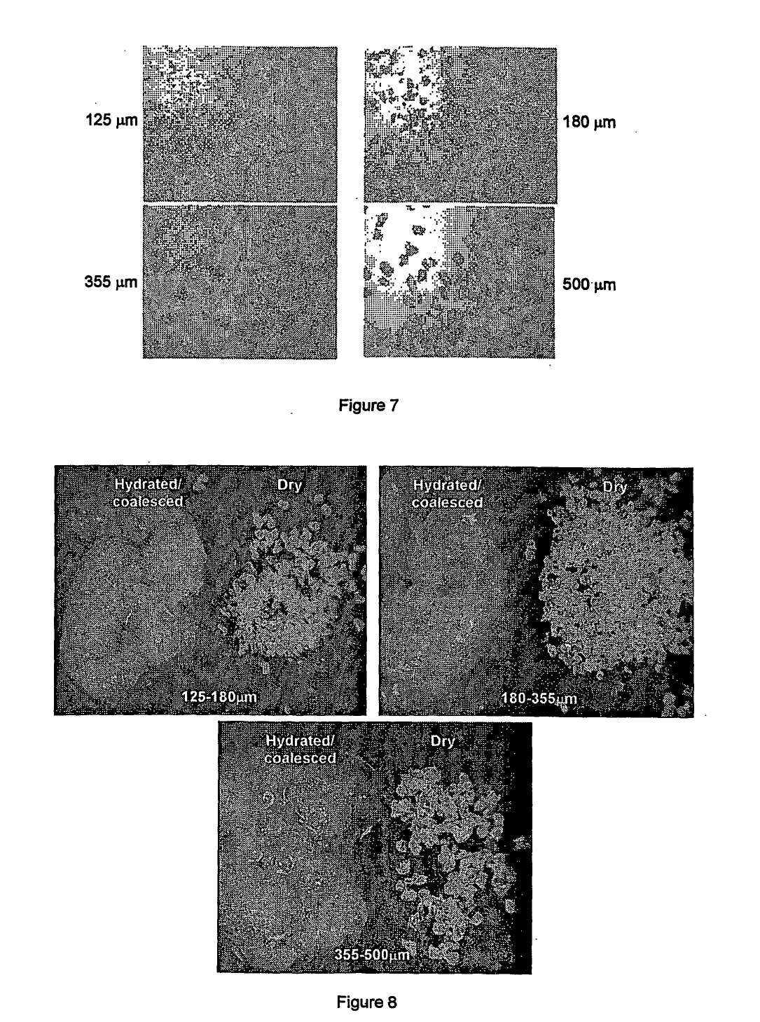

The gel was dehydrated at 120° C. overnight and the resulting xerogel milled into a range of particle sizes. The particles were sorted into size ranges by sieving as previously described in example 1. FIG. 7 shows micrographs of the xerogel particles after separation, the particles being those retained on the sieve of the specified aperture size.

Upon exposure to water, the particles swell rapidly. After some hours, the particles are seen to partially coalesce into a gel which has a water content in the region of 70%. FIG. 8 shows micrographs of the coalesced gels and the respective xerogels. For this type of hydrogel it will be necessary to hydrate the xerogels immediately before use and to deliver the composition before the gels coalesce such that deagglomeration upon gentle shaking does not happen.

### EXAMPLE 6

### PC-coated PVA Foam Particles for Embolisation

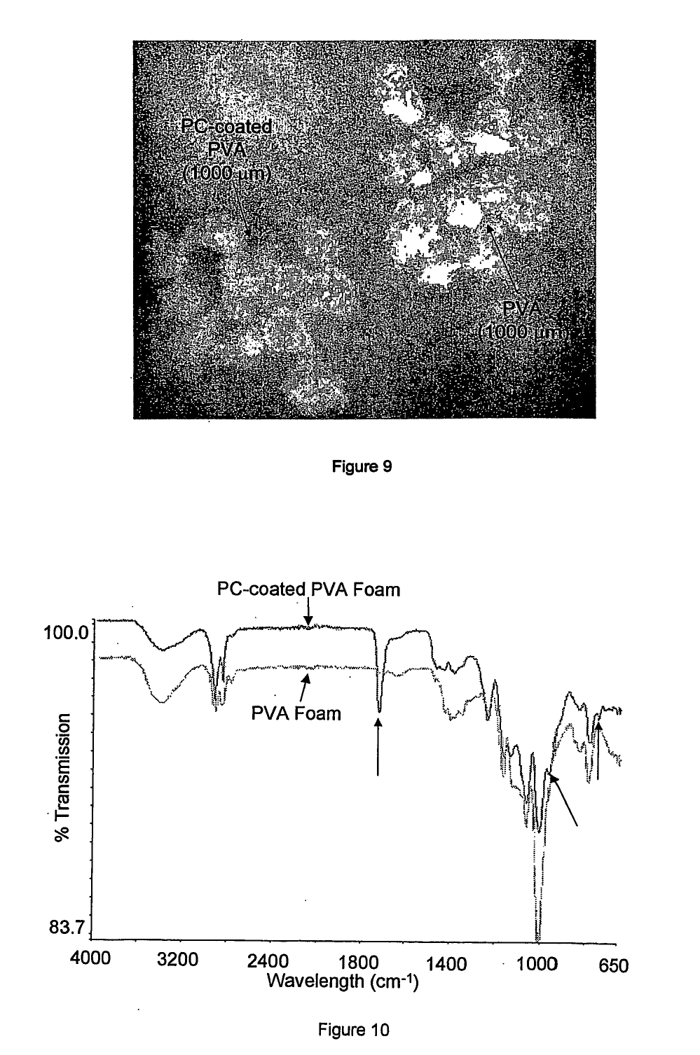

Into a 20 wt % chloroform solution of the crosslinkable polymer described in Reference Example 1, were placed polyvinyl alcohol (PVA) foam is particles (Cook Inc., USA) of mean size 1000 microns. After a few minutes, the solution was decanted and the particles allowed to air dry to remove the solvent. The particles were placed in an oven at 70° C. to effect curing of the coating. FIG. 9 shows the visual comparison of the particles before and after application of the coating. The size and shape of the particles is substantially unchanged, but the coating readers the surface somewhat shiny to the eye.

FIG. 10 compares the FT-IR spectra of an uncoated PVA foam particle with that of a PC-coated PVA foam particle. Note the additional intense peak at 1729 cm⁻¹ characteristic of the carbonyl of the ester linkages in the methacrylate PC polymer, and other signature peaks at 967 and 789 cm⁻¹ characteristic of the polymer and not the PVA. This demonstrated that the PVA particle was indeed coated successfully with the PC polymer.

### EXAMPLE 7

### PC-coated PVA Gelsphere for Embolisation

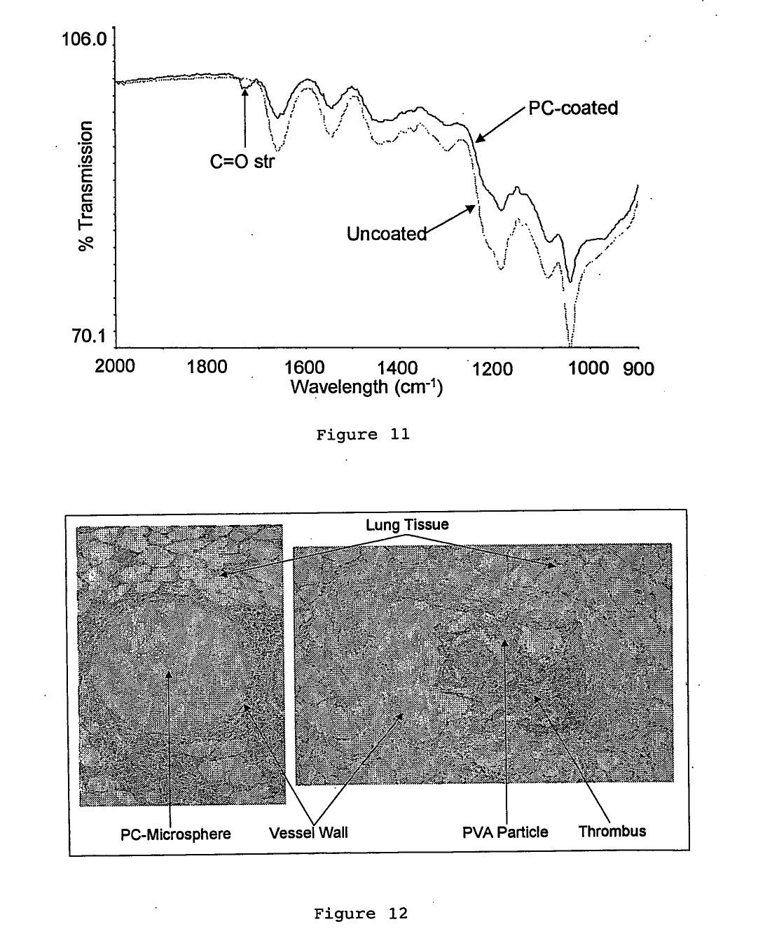

The same procedure as for example 6 was repeated using PVA Gelspheres (Biocure Inc produced according to WO-A-0168720, Example 2, but including an anionic comonomer, supplied as an aqueous composition with continuous aqueous liquid suspending swollen microspheres) that had been padded dry on absorbent paper but not dehydrated. The coating was crosslinked as before and the Gelsphere was observed to shrink to about 1/10^{th} its hydrated size during the curing step. FT-IR of the dehydrated sphere showed the presence of the carbonyl stretch characteristic of the PC coating that had been applied (FIG. 11). The coated sphere was seen to recover its original dimensions and shape upon hydration.

### EXAMPLE 8

### Assessment of Embolisation using PC-based Microspheres in a Sheep Lung Model

PC-based microspheres synthesised as described in example 3 were assessed compared to PVA particles (Cook Inc.) in a short-term sheep lung embolisation model.

Materials and Equipment

(i) Animals: 3 non-pregnant adult Pre-Alpes sheep
i. Weight range 40-60 kgs

(ii) PC-Spheres (as described in Example 3)

(iii) PVA-Particles (Cook Inc)

(iv) Qosina or BDH polypropylene syringes +3-way connectors

(v) Visipaque 320 contrast media

(vi) Terumo Leggiero diagnostic catheters

PC-spheres were dyed blue and were suspended in PBS (Inverclyde) in 5 ml syringes and sterilized in house by steam sterilization. PVA-particles were supplied sterile. Prior to each injection the mixture was agitated to maintain homogeneous particle suspension. The combined mixture was injected down microcatheters of a maximum 3-F diameter. Terumo Leggiero microcatheters were preferred because of their comparatively large internal lumen diameter.

Methods and Procedures

(i) Anesthesia

The sheep were not be fed for 24 hours prior to the procedure.

Anesthesia was induced by means of intramuscular injection of 15 mg/kg of body weight of Thiopental sodium (Nesdonal; Specia Rhone-Poulenc, Paris, France). Each animal was placed in a supine position, intubated, anesthetized with a mixture of 1.5% halothane (Trofiels, Zug, Switzerland) and 98.5% oxygen (CFPO, Paris, France) and ventilated with a unit (Logic 0.5; Ohmeda, Steeton, England). End-tidal CO₂ levels were measured continuously and maintained between 26 and 36 mm Hg with a monitor (N1000; Nellcor, Pleasanton, Calif.). Peripheral arterial oxygen saturation, maintained at a level higher than 95% was monitored with a probe applied to the ear. An electrocardiogram was used to continuously monitor each animal during the procedure.

(ii) Embolisation Procedure

Embolisation was performed using a venous femoral approach. A 4-or 5-F introducer sheath was placed into the vena cava. Retrograde catherisation of the vena cava was made to the right cavity of the heart and proximal catheterisation of the pulmonary artery. A 5-F guiding catheter was introduced and superselective cathetherisation of the pulmonary arterial branches. A control angiogram was taken, followed by embolisation in free flow of the particles (mixed with contrast). A repeat selective control angiogram was taken, followed by super selective embolisation. After each injection of sphere/particles, the catheter was purged with 2.5 ml of saline. Embolization was stopped when proximal arterial flow was reduced at angiographic evaluation. The same endpoint of embolisation was used for each sample.

Immediate Assessments
The following assessments were made immediately:—
Ease of application and handling
Effectiveness of embolisation (angiographic evaluation: level occluded, extent of arterial occlusion).
Endpoint
Volume of sphere/particle injected
Time to embolise vessel
Reflux or adverse effects at time of embolisation
Animal Weight: all animals will be weighed at the inclusion

Results

Both PC-microspheres and PVA particles embolised the vessels effectively. PVA tended to aggregate within the vessel and produce a more proximal occlusion, whereas the PC microspheres travelled more distally. PVA particles had a tendency to clump and aggregate within the catheter, whereas the compressible PC-microspheres had no issues with delivery. Both handled in an acceptable fashion for an embolisation agent. The blue PC-microspheres were easily seen in the syringe which made obtaining an isobouyant mixture with contrast easy to observe. Both PC spheres and PVA particles produced an acceptable endpoint for embolisation.

Histopathology

After 7 days the animals were sacrificed and a pathological and histological assessment performed on sections of the embolised lung tissue.

The lungs were fixed in 10% neutral buffered formalin by direct intratracheal injection and immersion and by staying in formalin for 2 weeks. Macroscopic examination was done in various lung areas. The samples were dehydrated in a series of alcohols and set in xylene, embedded in paraffin and cut into 3-4 μm sections. Each section was mounted on a microscope slide and stained with hematoxylin-eosin (H&E) stain and examined under light microscopy. Comparison of the PC-spheres with the PVA particles showed no unusual tissular reaction for the PC-microspheres. FIG. 12 shows sections from embolised vessels containing a PC-microsphere and PVA particle:

The section in FIG. 12 highlights the sphericity of the PC microsphere and how it conforms and maintains the natural shape of the vessel; this is in contrast to the irregular shaped PVA particle in which some of the space between particle and tissue is filled with thrombus.

1. A therapeutic or diagnostic composition comprising particles of a polymer matrix into which is absorbed aqueous liquid, the particles having diameters in the range 40 to 4000 μm, characterised in that surfaces of the particles express zwitterionic groups.

2. A composition according to claim 1 in which the polymer matrix is substantially non-biodegradable.

3. A composition according to claim 1 or claim 2 which comprises a continuous aqueous medium in an amount sufficient to suspend the particles.

4-33. (canceled)

34. A composition according to claim 1 which is sterile.

35. A composition according to claim 1 in which the zwitterionic group is ammonium, phosphonium, or sulphonium phosphate or phosphonate ester zwitterionic group.

36. A composition according to claim 5 in which the zwitterionic group has general formula III 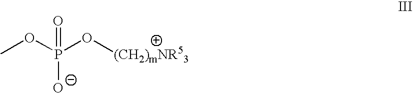
where the groups R⁵ are the same or different and each is hydrogen or C₁₋₄ alkyl, and m is from 1 to 4.

37. A composition according to claim 1 in which the zwitterionic groups are pendant groups on a polymer formed from ethylenically unsaturated monomers including a monomer of the general formula I
YBX I
in which Y is an ethylenically unsaturated group selected from the group consisting of H₂C═CR—CO-A-, H₂C═CR—C₆H₄-A¹-, H₂C═CR—CH₂A 2, R²O—CO—CR═CR—CO—O, RCH═CH—CO—O—, RCH═C(COOR²)CH₂—CO—O, 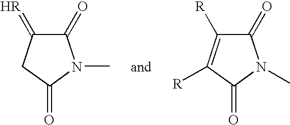
A is —O— or NR¹;
A¹ is selected from the group consisting of a bond, (CH₂)A² and (CH₂)₁ SO₃— in which I is 1 to 12;
A² is selected from the group consisting of a bond, —O—, O—CO—, CO—O, CO—NR¹—, —NR¹—CO, O—CO—NR¹-, NR¹—CO—O—;
R is hydrogen or C₁₋₄ alkyl;
R¹ is hydrogen, C₁₋₄ alkyl or BX;
R² is hydrogen or C₁₋₄ alkyl;
B is a bond, or a straight branched alkanediyl, alkylene oxaalkylene, or alkylene (oligooxalkylene) group, optionally containing one or more fluorine substituents;
X is the zwitterionic group.

38. A composition according to claim 37 in which Y is H₂C═CR—CO-A.

39. A composition according to claim 37 in which the ethylenically unsaturated monomers further comprise comonomers of the general formula V 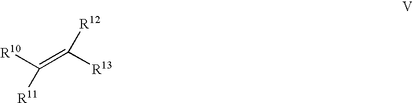
in which R¹⁰ is selected from the group consisting of hydrogen, halogen, C₁₋₄ alkyl and groups COOR¹⁴ in which R¹⁴ is selected from the group consisting of hydrogen and C₁₋₄ alkyl;
R¹¹ is selected from the group consisting of hydrogen, halogen and C₁₋₄ alkyl;
R¹² is selected from the group consisting of hydrogen, halogen, C₁₋₄ alkyl and groups COOR¹⁴ provided that R¹⁰ and R¹² are not both COOR¹⁴; and
R¹³ is selected from the group consisting of C₁₋₁₀ alkyl, C₁₋₂₀ alkoxycarbonyl, mono-or di-(C₁₋₂₀ alkyl) amino carbonyl, C₆₋₂₀ aryl, C₇₋₂₀ aralkyl, C₆₋₂₀ aryloxycarbonyl, C₁₋₂₀-aralkyloxycarbonyl, C₆₋₂₀ arylamino carbonyl, C₇₋₂₀ aralkyl-amino, hydroxyl and C₂₋₁₀ acyloxy groups, any of which may have one or more substituents selected from the group consisting of halogen atoms, alkoxy, oligo-alkoxy, aryloxy, acyloxy, acylamino, amino (including mono and di-alkyl amino and trialkylammonium in which the alkyl groups may be substituted), carboxyl, sulphonyl, phosphoryl, phosphino (including mono- and di-alkyl phosphine and tri-alkylphosphonium), zwitterionic, hydroxyl, vinyloxycarbonyl and other vinylic and allylic substituents, and reactive silyl and silyloxy groups;
or R¹³ and R¹² or R¹³ and R¹¹ may together form —CONR¹⁵CO in which R¹⁵ is a C₁₋₂₀ alkyl group.

40. A composition according to claim 39 in which the comonomer is a non-ionic comonomer.

41. A composition according to claim 1 in which the matrix polymer is a polyion complex.

42. A composition according to claim 1 in which the polymer matrix is covalently crosslinked.

43. A composition according to claim 42 in which the matrix polymer is formed from ethylenically unsaturated monomers including a di- or higher-valent ethylenically unsaturated monomer.

44. A composition according to claim 42 in which covalent crosslinking has been carried out by reaction of functional groups on preformed polymer subjected to conditions whereby intermolecular reaction takes place to form covalent bonds.

45. A composition according to claim 1 in which the matrix polymer is based on polyvinylalcohol.

46. A composition according to claim 45 in which the polyvinyl alcohol is crosslinked by reaction of pendant ethylenically unsaturated crosslinking groups by radical polymerisation.

47. A composition according to claim 1 in which the particles, when imbibed with physiological saline at room have a water content of at least 30% by weight.

48. A composition according to claim 1 in which the particles are substantially spherical.

49. A composition according to claim 1, in which the diameters of the particles, when fully imbibed with water, are in the range 150 μm to 3000 μm.

50. A composition according to claim 1 which is stable on storage at room temperature such that the particles do not coalesce to the extent that they cannot be redispersed upon gentle agitation.

51. Method of treatment of an animal in which a composition according to claim 1 is administered to an animal for therapy or diagnosis.

52. Method according to claim 51 in which the composition is administered to form an embolus.

53. Method according to claim 52 in which the composition is administered for uterine fibroid embolisation, embolisation of vessels around tumours or tumour-excision sites, embolisation of varicose veins or varicoceles, embolisation of arteriovenous malformations or venous malformations, hemostasis of gastro-intestinal bleeds, embolisation of fistulas or embolisation of fallopian tubes, or seminiferous tubes for sterilisation purposes.

54. Microspheres comprising a core which is a matrix of a water-insoluble water-absorbing polymer, which when imbibed with physiological saline at equilibrium at room temperature have diameters in the range 40 to 4000 μm, characterised by expressing zwitterions over their external surfaces.

55. Microspheres according to claim 54, which when fully imbibed with physiological saline have a water content of at least 30% by weight.

56. A kit comprising, each in separate vessels, a plurality of populations of microspheres according to claim 54 or 55, the populations differing in respect of the range of particle diameters.

57. A kit according to claim 56 in which the size range of each of the populations of microspheres, when imbibed with physiological saline to equilibrium at room temperature, is 50 to 1000 μm, preferably 150 to 300 μm.

58. A kit according to claim 57 in which the diameters of the different populations substantially do not overlap with one another.

59. A process of inverse suspension polymerisation in which ethylenically unsaturated monomers including a zwitterionic monomer of the general formula I
YBX I
in which Y is an ethylenically unsaturated group selected from the group consisting of H₂C═CR—CO-A-, H₂C═CR—C₆H₄-A¹-, H₂C═CR—CH₂A², R²O—CO—CR═CR—CO—O, RCH═CH—CO—O—, RCH═C(COOR²)CH₂—CO—O, 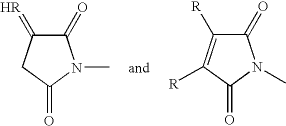
A is —O— or NR¹;
A¹ is selected from the group consisting of a bond, (CH₂)₁A² and (CH₂)₁ SO₃— in which I is 1 to 12;
A² is selected from the group consisting of a bond, —O—, O—CO—, CO—O, CO—NR¹—, —NR¹—CO, O—CO—NR¹—, NR¹—CO—O—;
R is hydrogen or C₁₋₄ alkyl;
R¹ is hydrogen, C₁₋₄ alkyl or BX;
R² is hydrogen or C₁₋₄ alkyl;
B is a bond, or a straight branched alkanediyl, alkylene oxaalkylene, or alkylene (oligooxalkylene) group, optionally containing one or more fluorine substituents; and
X is the zwitterionic group
and miscible comonomers, as a liquid monomer mixture, are dispersed into a continuous liquid non-solvent to form a dispersed phase, and initiator is added to initiate radical polymerisation in the dispersed phase, and the particles of polymer formed from the dispersed phase are recovered.

60. A process according to claim 59 in which the ethylenically unsaturated monomers include di- or higher functional crosslinking monomer.

61. A process according to claim 59 or claim 60 in which the monomer mixture comprises water.

62. A process according to claim 59 in which the liquid non-solvent comprises a water-in-oil stabiliser prior to addition of the monomer mixture.

63. A composition according to claim 35 in which the zwitterionic group is a group of the general formula II 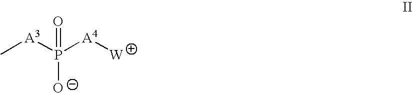
in which the moieties A³ and A⁴, which are the same or different, are —O—, —S—, —NH— or a valence bond, and W⁺ is a group comprising an ammonium, phosphonium or sulphonium cationic group and a group linking the anionic and cationic moieties which is a C₁₋₁₂-alkanediyl group,

64. A composition according to claim 63 in which W⁺ is a group of formula —W¹—N⁺R³₃, —W¹—P⁺R⁴₃, —W¹—S⁺R⁴₂ or —W¹-Het⁺ in which:
W¹ is alkanediyl of 2-6 carbon atoms optionally containing one or more ethylenically unsaturated double or triple bonds, disubstituted-aryl (arylene), alkylene arylene, arylene alkylene, or alkylene aryl alkylene, cycloalkanediyl, alkylene cycloalkyl, cycloalkyl alkylene or alkylene cycloalkyl alkylene, which group W¹ optionally contains one or more fluorine substituents and/or one or more functional groups; and
either the groups R³ are the same or different and each is hydrogen or alkyl of 1 to 4 carbon atoms, or aryl, or two of the groups R³ together with the nitrogen atom to which they are attached form an aliphatic heterocyclic ring containing from 5 to 7 atoms, or the three groups R³ together with the nitrogen atom to which they are attached as heteroaromatic ring having 5 to 7 atoms, either of which rings may be fused with another saturated or unsaturated ring to form a fused ring structure containing from 5 to 7 atoms in each ring, and optionally one or more of the groups R³ is substituted by a hydrophilic functional group, and
the groups R⁴ are the same or different and each is R³ or a group OR³, where R³ is as defined above; or
Het is an aromatic nitrogen-, phosphorus- or sulphur-, containing ring.

65. A composition according to claim 40 in which the nonionic comonomer is selected from the group consisting of C₁₋₂₄ alkyl(alk)-acrylates and -acrylamides, mono-, and di-hydroxy-C₁₋₆-alkyl(alk)-acrylates, and -acrylamides, oligo(C₂₋₃ alkoxy) C₂₋₁₈-alkyl (alk)-acrylates, -acrylamides, acrylamide, styrene, vinylacetate and N-vinyllactams.

66. A therapeutic or diagnostic composition comprising particles of a polymer matrix into which is absorbed aqueous liquid, the particles having diameters in the range 40 to 4000 μm, characterised in that surfaces of the particles express zwitterionic groups having general formula III 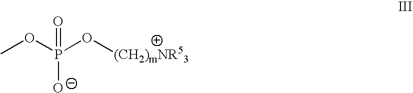
where the groups R⁵ are the same or different and each is hydrogen or C₁₋₄ alkyl, and m is from 1 to 4,
in which the matrix polymer is based on polyvinyl alcohol and the particles are substantially spherical.

67. A composition according to claim 66 in which the particles have diameters in the range 150 to 2000 μm when fully imbibed with water.

68. Microspheres according to claim 54 in which the zwitterions have general formula III 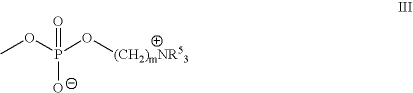
where the groups R⁵ are the same or different and each is hydrogen or C₁₋₄ alkyl, and m is from 1 to 4.

69. Microspheres according to claim 54 in which the polymer is based on polyvinyl alcohol.
